(19) United States
(12) Patent Application Publication
(10) Pub. No.: US 2010/0292543 A1
(43) Pub. Date: Nov. 18, 2010

(54) Analysis of Endogenous Fluorescence Images to Extract Morphological/Organization Information About Living Samples

(75) Inventors: Jonathan M. Levitt, Cranston, RI (US); Claudia Mujat, Acton, MA (US); Martin Hunter, Somerville, MA (US); Irene Georgakoudi, Acton, MA (US)

Correspondence Address: PROSKAUER ROSE LLP, ONE INTERNATIONAL PLACE, BOSTON, MA, 02110, US

(73) Assignee: Tufts University, Medford, MA (US)

(21) Appl. No.: 12/739,271

(22) PCT Filed: Oct. 30, 2008
(86) PCT No.: PCT/US08/81894

§ 371 Date: Jul. 9, 2010

Related U.S. Application Data
(60) Provisional application No. 60/983,900, filed on Oct. 30, 2007.

(51) Int. Cl. A61B 5/00; G06K 9/00

(52) U.S. Cl. 600/300; 382/133

(57) ABSTRACT

Methods and computer program products for analyzing tissue are provided. The tissue is exposed to light at the appropriate wavelengths for inducing fluorescence. Images of the fluorescing tissue are taken at two or more depths within the tissue. The PSD function is determined for each image at a different depth within the tissue. A characteristic of each PSD function determined is compared, and it is determined whether or not the tissue exhibits a pre-cancerous characteristic.

### GOVERNMENT SUPPORT

The invention described here in was supported in whole or in part by Grant No. R01 CA097966 from the National Institutes of Health. The Government has certain rights in the invention.

### BACKGROUND

A number of techniques, which hold the promise of truly non-invasive, accurate and rapid detection of pre-cancerous and cancerous lesions, take advantage of alterations in optical signatures induced by either biochemical or morphological changes in diseased cells (I. Georgakoudi, J. Motz, V. Backman, G. Angheloiu, A. Haka, M. Muller, R. Dasari, and M. S. Feld, Quantitative characterization of biological tissue using optical spectroscopy,” in Biomedical Photonics Handbook, T. Vo-Dinh, ed. (CRC Press, 2003), pp. 1-33.). These alterations are often the result of an imbalance in cell differentiation, proliferation and programmed cell death pathways. NADH and FAD autofluorescence along with the corresponding redox ratio has led the way to the use of autofluorescence for the acquisition of important biochemical information, such as metabolic status of cells and tissues. (B. Chance, P. Cohen, F. Jobsis, and B. Schoener, Science 137 (3529), 499 (1962)).

### SUMMARY OF THE INVENTION

Provided herein are methods and computer program products for analyzing high resolution optical images of cellular fluorescence of a tissue sample. The information acquired can be used to assess morphological and organizational aspects of the tissue sample at the cell and tissue level. The methods and computer program products provided herein can be used, for example, to analyze a tissue for the presence of pre-cancerous characteristics. For example, as described herein, the power spectral density (PSD) of depth-resolved two-photon excited fluorescence (TPEF) images of NADH can be used to reveal significant differences between normal and pre-cancerous tissues. Thus, the methods and computer program products provided herein could be used to assess both biochemical and structural tissue features with significant diagnostic potential.

Methods for analyzing a tissue are provided. In some embodiments, the tissue has at least two layers of cells. In some embodiments, the method comprises determining the PSD function at a designated range of spatial frequencies for each of two or more images of the tissue. In some embodiments, the PSD function is determined using high resolution images of the tissue. In some embodiments, the high resolution images are obtained at different depths within the tissue. The PSD functions are compared. In some embodiments, the PSD functions or one or more characteristics of the PSD functions corresponding to two or more depths within the tissue are compared.

In other embodiments of the method, high resolution fluorescence images of the tissue are obtained. High resolution fluorescence images of the tissue are obtained at two or more depths within the tissue. The PSD function for a designated range of spatial frequencies is obtained for the image(s) at each depth, and the PSD functions are compared.

Methods for determining whether a tissue exhibits a pre-cancerous characteristic are provided. In some embodiments, the method comprises determining the PSD function at a designated range of spatial frequency for each of two or more images of the tissue. In some embodiments, the high resolution images are obtained at different depths within the tissue. The PSD functions are compared. In some embodiments, the PSD functions or one or more characteristics of the PSD functions corresponding to two or more depths within the tissue are compared, thereby determining whether the tissue exhibits a pre-cancerous characteristic.

In other embodiments of the method, high resolution fluorescence images of the tissue are obtained at two or more depths within the tissue. The PSD function for a designated range of spatial frequencies is obtained for the image(s) at each depth. The PSD functions are compared, thereby determining whether the tissue exhibits a pre-cancerous characteristic.

Computer program products for analyzing a tissue are also provided. In some embodiments, a computer program product is tangibly embodied in an information carrier. The computer program product includes operable instructions that cause a data processing apparatus to determine the PSD function at a range of spatial frequencies for one or more high resolution images. The computer program product instructs the data processing apparatus to compare the PSD functions obtained from the images taken for example, at different depths. In some embodiments, the high resolution images are obtained at a different depth within a tissue having at least two layers of cells.

In some embodiments, the computer program product also includes operable instructions that cause a data processing apparatus to obtain high resolution fluorescence images of a tissue at two or more depths within the tissue. The computer program product instructs the data processing apparatus to determine the PSD functions at a range of spatial frequencies corresponding to subcellular matter. The computer program product causes the data processing apparatus to compare a characteristic of each PSD function obtained.

The various embodiments described herein can be complimentary and can be combined or used together in a manner understood by the skilled person in view of the teachings contained herein.

### BRIEF DESCRIPTION OF THE DRAWINGS

FIG. 1 shows PSD depth profiles for a single HFK (A) and HPV (B) raft, and depth variance from multiple rafts (C).

FIG. 2 shows NADH TPEF depth images from a single HFK raft at the superficial (A), intermediate (B), and basal layers (C), and corresponding PSD calculations with power law fit and residuals (D-F).

FIG. 3 shows NADH TPEF depth images from a single HPV raft at the superficial (A), intermediate (B), and basal layers (C), and corresponding PSD calculations with power law fit and residuals (D-F).

### DETAILED DESCRIPTION OF THE INVENTION

As demonstrated herein a self-affine spatial distribution of mitochondrial NADH at length scales 1-10 μm is indicated by the inverse power law PSD behavior of NADH autofluorescence intensity images from normal and pre-cancerous and cancerous engineered epithelial tissues. There are significant differences in PSD inverse power law exponents as a function of tissue depth, between normal and pre-cancerous skin models, suggesting changes in cellular differentiation and fractal organization of mitochondria with the onset of pre-cancerous lesions. In addition, marked differences in the PSD functions are observed in the low frequency range (κ<0.1 μm⁻¹).

Methods for analyzing a tissue or tissue sample are provided. In some embodiments, the tissue or tissue sample comprises two or more layers of cells. In some embodiments, the method comprises determining the PSD function at a designated range of spatial frequencies for each of two or more images of the tissue. In some embodiments, the PSD function is determined using high resolution images of the tissue. In some embodiments, the high resolution images are obtained at different depths within the tissue. The PSD functions are compared. In some embodiments, the PSD functions or one or more characteristics of the PSD functions corresponding to two or more depths within the tissue are compared. In some embodiments, the high resolution images are provided to the user for analysis according to the methods provided herein. In other embodiments, the high resolution images are obtained by the user as part of the method.

To obtain images, the tissue or sample of tissue can be exposed to light such that fluorescence is induced. In some embodiments, the tissue or tissue sample is excited using any suitable high resolution technique, such as multi-photon excitation. Multi-photon excitation includes, for example, two-photon excitation or three-photon excitation.

The tissue may be excited by exposure to light having a wavelength specifically chosen to excite a chosen molecule or component within the cell. For example, the wavelength can be chosen to excite NADH or FAD though the chosen components for excitation are not limited to these substances.

Typically, NADH and FAD are found in the mitochondria. Therefore, a wavelength chosen to excite NADH or FAD can be used to detect mitochondria within the cells. The tissue may be exposed, for example, to light having a wavelength between 700 and 900 nm. The tissue may be exposed to light having a wavelength at about 740 nm. The tissue may be exposed to light having a wavelength of about 800 nm. Light having the desired wavelength can be generated by any suitable method known in the art. For example, a particular lamp that produces the desired wavelength can be used in combination with one or more filters that allows light of the appropriate wavelength to reach the tissue or sample of tissue during imaging.

The fluorescence induced may be autofluorescence or endogenous fluorescence. In one potential embodiment of the method, autofluorescence is induced in the tissue through two-photon excitation at a wavelength of 740 nm. In other embodiments, the tissue or sample of tissue can be labeled with a fluorescent dye. A fluorescent dye can be used that specifically labels a subcellular structure such as mitochondria, nuclei, endoplasmic reticulum, and the like. Suitable fluorescent dyes are known in the art. For example, fluorescent dyes that label mitochondria include compounds that stain the mitochondria of living cells such as nonyl acridine orange, rhodamine 123, and dihydrorhodamine 123. Fluorescent dyes that stain and are retained by mitochondria even after fixation of the stained cells can also be used.

The tissue is illuminated with light comprising a suitable wavelength and two or more high resolution images of the tissue are obtained. Two or more images are obtained at each of two or more depths in the tissue or tissue sample. In some embodiments, the different depths correspond to different cellular layers within the tissue.

Where images are to be obtained by the user, the image capturing apparatus may be any suitable apparatus for capturing a high resolution image. For example the apparatus can be a confocal microscope. In some embodiments, the image capturing apparatus has a resolution of 1 μm or better (e.g., objects smaller than 1 μm can be resolved). Images may be taken at successively different depths within the tissue. Images may also be taken at uniform or non-uniform increments of depth within the tissue. In one embodiment of the method, images are taken at 1 μm increments at successively deeper depths into the tissue, for example, from the superficial layer to the basal layer. In another embodiment, images may be taken up to a few hundred microns into the tissue.

According to the methods provided herein, high resolution images, either provided to the user or obtained by the user are analyzed by determining the PSD function Φ(κ) of the images at a designated range of spatial frequencies. In some embodiments, the PSD function obtained is the radial, angle-averaged PSD.

The range of spatial frequencies determines the type of information the PSD function reveals about the tissue. At high spatial frequencies, ranging for example, from 0.3 to 1 μm⁻¹ the PSD function provides information about subcellular matter in the tissue. Subcellular matter includes, for example, mitochondria. At lower spatial frequencies, ranging, for example, from 0.05 to 0.1 μm⁻¹, the PSD function provides information about nuclear matter in the tissue. At low spatial frequencies, for example, below 0.05 μm⁻¹, the PSD function provides information about intercellular matter in the tissue. The PSD function of an image may be taken at any or all of these ranges of spatial frequencies to capture information about the tissue at the subcellular, nuclear, and intercellular levels.

According to the methods provided herein, a characteristic of each PSD function obtained is compared with the a characteristic of one or more of the other PSD functions obtained. In this manner, where images are obtained at different depths within the tissue, the PSD function provides information about cellular morphology and structure throughout the tissue or tissue sample. Depending on the designated range of spatial frequency or frequencies used, the characteristic of the PSD function provides information regarding the subcellular, nuclear, and/or intercellular morphology and structure of the cells of the tissue. In some embodiments, whether the tissue exhibits a pre-cancerous or cancerous characteristic is determined based on the chosen characteristic of the PSD function.

One characteristic of the PSD function to compare is variance in the function at different depths of tissue. Comparing the variance of the PSD functions obtained from images at different depths provides information about differentiation within the tissue. The type of comparison performed may depend on whether the subcellular, nuclear, or intercellular properties of the tissue are being examined.

Regarding variance of PSD function with the depth of tissue, as demonstrated herein, as normal cells differentiate, the subcellular components typically become more organized (or self-affine). Pre-cancerous or cancerous cells appear less developed and show less organized morphology. Thus, the variance of the PSD function with depth at high spatial frequencies may be compared to gain information about the tissue. If the variance of the PSD function varies with depth, the tissue is scored as negative for that pre-cancerous or cancerous characteristic. However, if the variance does not vary significantly with depth, the tissue is scored as having a pre-cancerous or cancerous characteristic.

Another such characteristic of the PSD function is the power exponent, α. At high spatial frequencies, which correspond to subcellular components, the PSD functions of normal, pre-cancerous, and cancerous tissues demonstrate a consistent inverse power law dependence, Φ(κ)∝κ^{-α}. The relationship suggests that subcellular components such as mitochondria exhibit self-affine fractal organization. As demonstrated herein the power exponent α is related to the self-affine fractal correlations in the spatial distribution of subcellular components, and it is thus related to the level of randomness in the organization of the subcellular components. Thus, in some embodiments, the PSD functions at high spatial frequencies are obtained for high resolution images corresponding to different depths within the tissue. The power exponents of the PSD functions are determined and compared.

Without wishing to be bound by theory, it is thought that normal cells differentiate as they grow and migrate from the basal layer to the superficial layer in a stratified tissue or tissue sample. The differentiation typically changes the cell from a small, uniform shape having a relatively large nucleus to cytoplasm ratio to a larger, more amorphous shape having a smaller nucleus to cytoplasm ratio. On the other hand, pre-cancerous or cancerous cells are thought not to differentiate as they migrate. Pre-cancerous and cancerous cells typically resemble undifferentiated cells of the cell lineage. Consequently, as demonstrated herein the spatial organization and extent of random behavior exhibited by subcellular components differs among different layers of normal tissue, as revealed by the PSD function. However, as demonstrated herein, the spatial organization and extent of random behavior exhibited by subcellular components are more similar among the different depths of pre-cancerous tissue. Thus, the power exponent varies according to depth of normal tissues, while the power exponent varies less across depth in pre-cancerous or cancerous tissue. Therefore, in the methods and computer program product provided herein, if the power exponent is found to vary little across depth, the tissue is scored as positive for a pre-cancerous characteristic. If the power exponent is found to vary significantly across depth, the tissue is scored as negative for that pre-cancerous characteristic.

Regarding nuclear-sized components, as normal cells differentiate, the ratio of the cytoplasm to the nucleus typically increases. In pre-cancerous or cancerous cells, the ratio of the nucleus to cytoplasm typically remains higher than that of a differentiated cell of the same lineage. Thus, the variance of the PSD function with depth at low spatial frequencies may be compared with the variance of the PSD function with depth at higher and lower spatial frequencies, at different depths within the tissue. If the variance of the PSD function at the spatial frequencies corresponding to nuclear-sized components increases compared to the variance at surrounding spatial frequencies, the tissue is scored as not having a pre-cancerous or cancerous characteristic. However, if the variance does not change significantly, the tissue is scored as having a pre-cancerous or cancerous characteristic.

Regarding intercellular organization, the variance of the PSD function with depth at low spatial frequencies corresponding to intercellular organization may be compared. If the variance of the PSD function varies with depth, the tissue is scored as negative for that pre-cancerous or cancerous characteristic. However, if the variance does not change significantly with depth, the tissue is scored as having a pre-cancerous or cancerous characteristic.

The methods provided herein may be performed on any number of tissue sample, tissues, or organs, including but not limited to the oral cavity, cervix, lung, bronchus, breast, esophagus, colon, bladder, gastrointestinal tract, ureters, skin, bile ducts, pancreatic ducts, liver, or prostrate. Suitable cells include epithelial cells having two or more layers, wherein one of the layers is a basal layer.

The method may be performed on tissue that has been extracted from a patient. Tissue that has been extracted may be obtained by any suitable method known in the art for obtaining tissue from a patient, for example, through a biopsy. The tissue may be an incisional, core, or excisional biopsy. The tissue obtained through excisional biopsy may be a resection. The tissue may be obtained through needle aspiration biopsy. The extracted tissue may be obtained in the same facility that performs the analysis. For example, the tissue may be obtained at the point of care, for example, in a hospital or clinic, and sent to an on-site laboratory for analysis. Alternatively, the extracted tissue may be obtained at the point of care and transported to a facility that performs the analysis.

Any suitable method for preserving or maintaining and transporting biopsy tissue may be used. In some embodiments, the tissue sample is perfused such that the cells remain intact and/or viable. The tissue sample may be treated such that the cells remain intact and viable during the imaging. In other embodiments, the tissue sample can be fixed such that the cells are not necessarily viable, but such that the tissue sample is suitable for the high resolution fluorescence imaging described herein. As described herein, the tissue may be labeled with a fluorescent dye that is capable of staining a cellular structure of component of interest such that the stained structure can be analyzed using high resolution fluorescence imaging.

Tissue may also be accessed in situ. For example, tissue may be accessed by inserting an endoscope into an anatomical cavity. The endoscope may be manipulated mechanically or electronically towards the tissue site of interest. Narrow caliber endoscopes may be passed through the biopsy channels of larger endoscopes to obtain cellular fluorescence imaging from organs. Narrow caliber endoscopes may also be passed through a large bore needle or trocar to examine solid organs. The endoscope may emit light to induce fluorescence in the tissue, and it may capture images. The captured images are analyzed as described herein.

Computer program products are also provided. In some embodiments, the computer program product is stored on a computer usable medium. A computer usable medium can include a readable memory device, such as a hard drive device, CD-ROM, a DVD-ROM, or a computer diskette, having computer readable program code segments stored thereon. The computer readable medium can also include a communications or transmission medium, such as, a bus or a communication link, either optical, wired or wireless having program code segments carried thereon as digital or analog data signals. The computer program products provided herein include operable instructions that cause a data processing apparatus to determine the PSD function at a range of spatial frequencies for one or more high resolution images. The computer program product instructs the data processing apparatus to compare the PSD functions obtained. In some embodiments, the high resolution images were obtained at a different depth within a tissue having at least two layers of cells.

In some embodiments, the computer program product also includes operable instructions that cause a data processing apparatus to obtain high resolution fluorescence images of a tissue at two or more depths within the tissue. The computer program product instructs the data processing apparatus to determine the PSD functions at a range of spatial frequencies corresponding to subcellular matter for images at a plurality of depths. The computer program product causes the data processing apparatus to compare a characteristic of each PSD function obtained at a plurality of depths.

### Exemplification

As described herein, depth-resolved NADH autofluorescence images differentiate between normal and pre-cancerous or cancerous engineered tissues. An inverse power law behavior of the PSD of these images was observed, indicating a self-affine organization of mitochondrial NADH at length-scales 1-10 μm. Power exponents of the PSD functions vary significantly with tissue depth and pre-cancerous state, giving insight into the morphological changes associated with pre-cancerous lesions and providing substantial potential for non-invasive clinical diagnosis of squamous epithelial lesions and tumors.

NADH TPEF images were acquired with a Leica TCS SP2 spectral confocal microscope equipped with a Ti:Sapphire laser (Mai Tai, Spectra Physics/Oriel) providing 100 fs pulses at 80 MHz. Samples were excited at 740 nm using a water immersion 60× (1.2 NA) objective. TPEF emission was excited using a descanned PMT detector after passing through a 700 nm short pass filter, a 495 dcxr dichroic and a 455±35 nm bandpass filter. Images acquired from engineered epithelial tissues constructed with normal human foreskin keratinocytes (HFK) and with human papillomavirus (HPV)-immortalized epithelial cells were analyzed. The engineered tissues were set up as described by C. Meyers, T. J. Mayer, and M. A. Ozbun, J. Virol. 71 (10), 7381 (1997). Briefly, normal or HPV-immortalized keratinocytes (K. E. Creek, G. Geslani, A. Batova, and L. Pirisi, Adv. Exp. Med. Biol. 375, 117 (1995).) were seeded on the surface of a matrix consisting of type I collagen with embedded fibroblasts. (C. Meyers, T. J. Mayer, and M. A. Ozbun, J. Virol. 71 (10), 7381 (1997).) Once the cells had formed a near confluent monolayer, the collagen blocks were lifted so that the keratinocytes were at the air-liquid interface. After ten days of culture, the keratinocytes formed multilayered skin-like structures which were subjected to imaging. The NADH TPEF fluorescence images were quantified by calculating the radial, angle-averaged PSD throughout at 1 μm depth increments throughout the tissue.

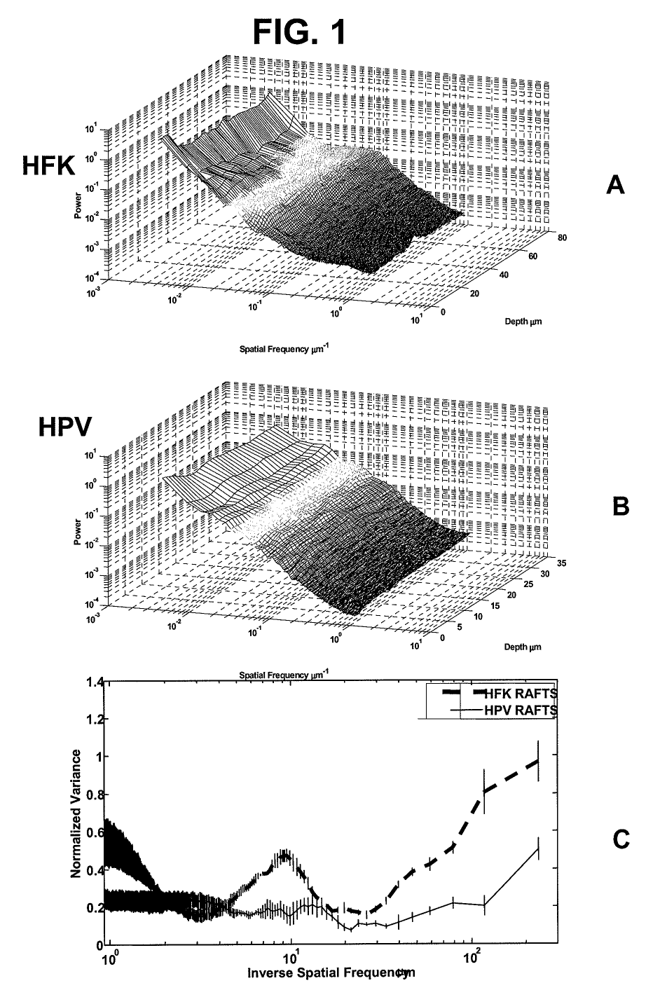

FIG. 1 shows the resulting PSD functions, Φ(κ), obtained as a function of tissue depth from normal and model pre-cancerous engineered tissues. A striking difference between the two samples is the greater variance of the normal PSD functions with depth at the subcellular, nuclear and intercellular level (FIG. 1C). At high spatial frequencies (0.3<κ<1 μm⁻¹), the PSD functions from the pre-cancerous model tissue are almost invariant with depth, whereas the normal model tissue shows a clear increase in PSD variance with spatial frequency. A prominent increase in PSD variance of the normal tissue is also observed around κ=0.1 μm⁻¹, corresponding to changes in the morphology of nuclear-sized features. Larger PSD fluctuations are clearly observed in samples of normal model tissues at low spatial frequencies (κ<0.05 μm⁻¹), indicating greater changes in their intercellular organization with tissue depth.

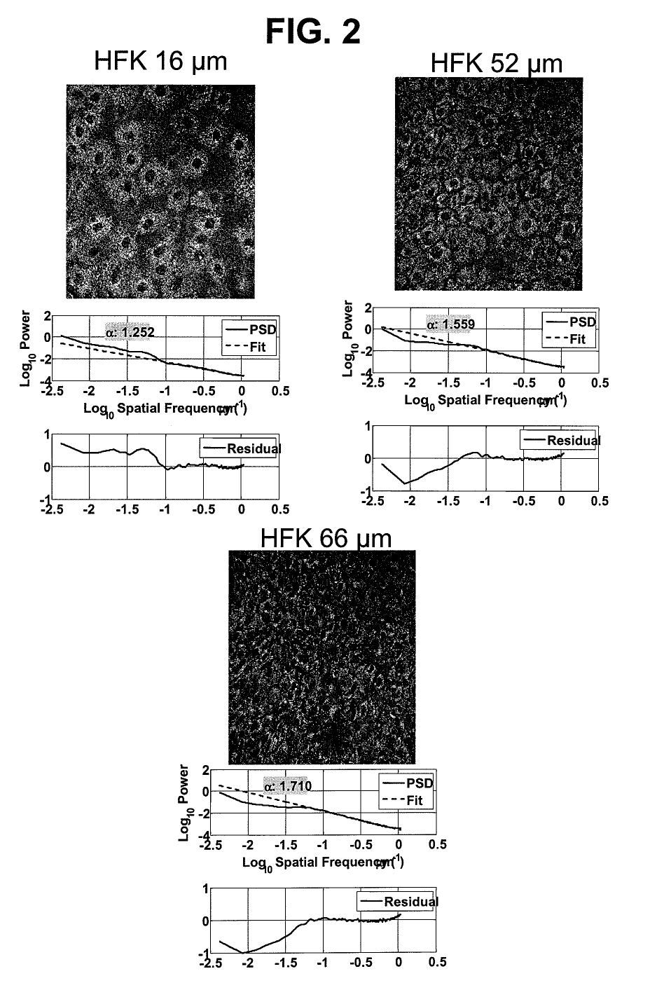

A closer look at the PSD functions at high spatial frequencies reveals a consistent inverse power law dependence, Φ(κ)∝κ^{-α}, for both normal and pre-cancerous model tissues. FIG. 2 shows select NADH autofluorescence images from an engineered normal epithelial tissue, at depths of 16, 52 and 66 μm (superficial, intermediate and basal layers, respectively), and their corresponding PSD functions fitted for inverse power law behavior in the spatial frequency range 0.1<κ<1.0 μm⁻¹. Power law scaling of Φ(κ) is observed at all tissue depths, with power exponents a varying strongly (increasing) with tissue depth (Table 1). By contrast, the high frequency power scaling of Φ(κ) for the pre-cancerous model tissue is almost invariant with tissue depth.

The inverse power law dependence of Φ(κ) at high spatial frequencies (0.1<κ<1.0 μm⁻¹) can be attributed to a self-affine fractal organization of the mitochondrial NADH at length-scales 1.0-10 μm. A statistically self-affine function, ƒ(χ), is one whose variance, S(χ), is given by S(χ)=<|ƒ(χ+α)−ƒ(χ)|²>∝|α|^{2H}, where the Hurst parameter, H, is limited to the range 0<H<1. For a self-affine function in E-dimensional Euclidean space, the PSD along any straight line path in E-space is an inverse power law, Φ(κ)∝κ^{-α}, and its fractal dimension, D, is given by D=E+1−H=E+½(3−α). (R. F. Voss, Physica Scripta T13, 27 (1986)). The fractal parameters thus derived for the normal and pre-cancerous tissues depicted in FIGS. 1-3 are listed in Table 1.

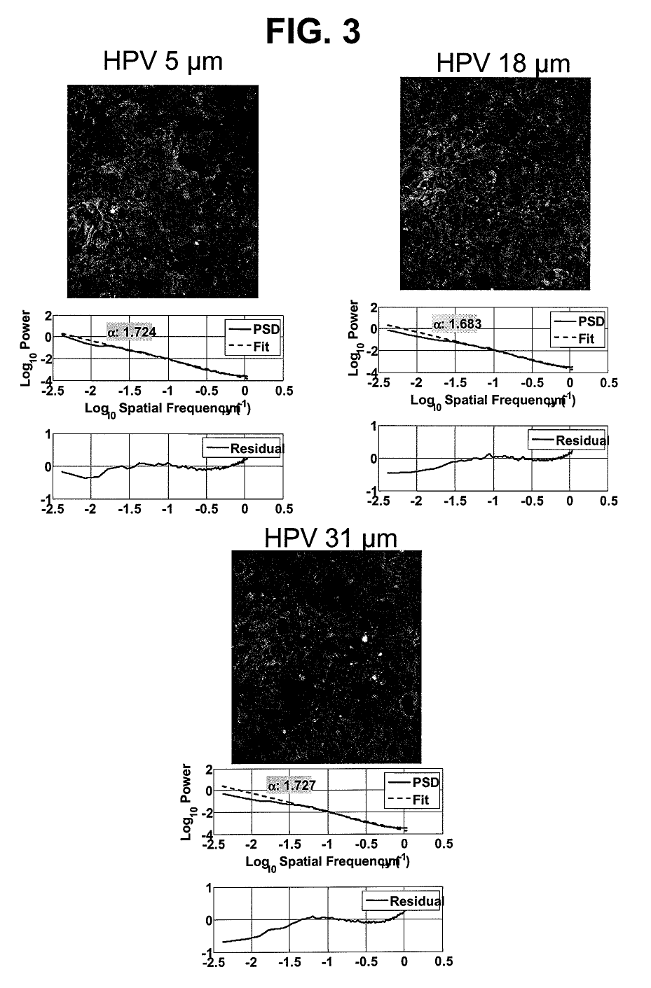

Self-affine functions are also known as fractional Brownian functions, given their close association with random walk statistics. (B. B. Mandelbrot, The Fractal Geometry of Nature (W.H. Freeman & Co., New York, 2000)). A value of H=0.5 corresponds to exact Brownian behavior, with a Gaussian distribution of increments ƒ(χ+α)−ƒ(χ) i.e., for any three spatial positions χ₁<χ<χ₂, ƒ(χ)−ƒ(χ₁) is statistically independent of ƒ(χ₂)−ƒ(χ). (R. F. Voss, Physica Scripta T13, 27 (1986)). For values of H>0.5, the increments ƒ(χ+α)−ƒ(χ) become positively correlated, while for H<0.5 they exhibit negative correlations. The results provided herein indicate that the spatial organization of mitochondrial NADH is statistically self-affine and negatively correlated (H<0.5). Furthermore, a distinct gradient in the fractal dimension of the normal engineered tissues was observed as a function of depth, with significantly higher Hurst parameters for the basal layer (H_{b}=0.36) compared to the intermediate and superficial layers of normal tissue (Hᵢ=0.28 and Hₛ=0.13). By contrast, no significant variation of H was observed in the pre-cancerous model tissue, where H was consistently close to the value for the basal layer in engineered normal tissue (H_{b}=0.37, Hᵢ=0.34 and Hₛ=0.36). A plausible interpretation is that as normal epithelial cells migrate to the surface and differentiate, there is a trend towards higher spatial organization (less random character) of the mitochondrial NADH, evidenced by the values of H progressively lower than H=0.5 (Table 1). In engineered pre-cancerous tissue, on the other hand, the value of H remains similar to that of the undifferentiated, normal basal cell layer throughout the full thickness of the engineered skin. This is supported by the lack of visual differentiation apparent in FIGS. 3A-C, compared to that in FIGS. 2A-C.

As demonstrated herein, mitochondrial NADH distribution in normal basal and intermediate epithelium shows negatively-correlated self-affine fluctuations. Furthermore, as demonstrated herein, NADH autofluorescence PSD functions also show significant features at low spatial frequencies. This is most evident in the superficial layer of normal tissue models, which show a prominent peak in the region 0.04<κ<0.08 μm⁻¹ associated with the cell nuclear perimeters, which are highlighted in the images by the lack of intranuclear NADH (FIG. 2D).

There are numerous ways to use the method. A patient visits a hospital for an endoscopic procedure. During the procedure, a doctor or technician inserts an endoscope into the body cavity to examine the tissue of interest. The doctor or technician controls the endoscope to emit light that causes the mitochondrial NADH of the epithelial cells to fluorescence. High resolution images are taken at 1 μm increments to a depth of 300 μm or in general to the depth of the basal cell layer. The images can be analyzed at the point of care, or the images can be sent to another location for analysis. The analysis includes, for example, obtaining the PSD functions at a designated range of spatial frequencies for each image. Characteristics of the PSD function are compared to determine if the tissue examined exhibits a pre-cancerous characteristic.

Alternatively, a surgeon might perform surgery to excise tissue from the patient. The excised tissue is analyzed as described herein. For example, the tissue could be sent to a laboratory where the tissues would be illuminated so that the tissue fluoresces, and images taken, for example, using a confocal microscope. Images could be taken, for example, at increments of 3 μm to a depth of 300 μm or the depth at which the basal layer of cells is present. PSD functions at a designated range of frequencies would be obtained for each image, and characteristics of the PSD functions would be compared to determine if the tissue exhibits a pre-cancerous characteristic.

The methods and computer program products provided herein may be embodied in other specific forms without departing from the spirit or essential characteristics thereof. The foregoing embodiments are therefore to be considered in all respects illustrative rather than limiting on the methods and computer program products described herein. Scope of the invention is thus indicated by the appended claims rather than by the foregoing description, and all changes which come within the meaning and range of equivalency of the claims are therefore intended to be embraced therein.

We claim:

1. A method for analyzing tissue having at least two layers of cells comprising:
(a) determining a power spectral density (PSD) function at a designated range of spatial frequencies for each of two or more images of the tissue, wherein each image is obtained at a different depth within the tissue, the PSD function being determined using high resolution fluorescence images of the tissue, and
(b) comparing the PSD functions obtained.

2. The method of claim 1, wherein the two or more depths correspond to different cellular layers within the tissue.

3. The method of claim 1, wherein the high resolution fluorescence is multi-photon excited fluorescence.

4. The method of claim 1, wherein the high resolution fluorescence is two-photon excited fluorescence.

5. The method of claim 1, wherein the high resolution of fluorescence is 1 micron or less.

6. The method of claim 1, wherein the fluorescence is autofluorescence.

7. The method of claim 1, wherein comparing the PSD functions comprises comparing a characteristic of each PSD function.

8. The method of claim 7, wherein the characteristic of the PSD function is a power exponent α.

9. The method of claim 7, wherein the characteristic of the PSD function is variance across depth.

10. The method of claim 1, wherein the spatial frequency range corresponds to tissue matter.

11. The method of claim 1, wherein the spatial frequency corresponds to nuclear or intercellular matter.

12. The method of claim 1, wherein the spatial frequency range corresponds to subcellular matter.

13. The method of claim 12, wherein the subcellular matter is mitochondria.

14. The method of claim 1, wherein the images are obtained by exposing the tissue to light having a wavelength between 700 and 900 nm.

15. The method of claim 12, wherein the images are obtained by exposing the tissue to light having a wavelength of about 740 nm.

16. The method of claim 12, wherein the images are obtained by exposing the tissue to light having a wavelength of about 800 nm.

17. A method for determining whether tissue exhibits a pre-cancerous characteristic comprising:
(a) determining a power spectral density (PSD) function at a designated range of spatial frequencies for each of two or more images of the tissue, wherein each image is obtained at a different depth within the tissue, the PSD function being determined using high resolution fluorescence images of the tissue, and
(b) comparing the PSD functions obtained.

18. The method of claim 17, wherein the two or more depths correspond to different cellular layers within the tissue.

19. The method of claim 17, wherein the high resolution fluorescence is multi-photon excited fluorescence.

20. The method of claim 17, wherein the high resolution fluorescence is two-photon excited fluorescence.

21. The method of claim 17, wherein the high resolution of fluorescence is 1 micron or less.

22. The method of claim 17, wherein the fluorescence is autofluorescence.

23. The method of claim 17, wherein comparing the PSD functions comprises comparing a characteristic of each PSD function.

24. The method of claim 23, wherein similar characteristics at each depth indicate whether the tissue exhibits a pre-cancerous characteristic.

25. The method of claim 23, wherein the characteristic of the PSD function is a power exponent α.

26. The method of claim 23, wherein the characteristic of the PSD function is variance across depth.

27. The method of claim 17, wherein the spatial frequency range corresponds to tissue matter.

28. The method of claim 17, wherein the spatial frequency range corresponds to subcellular matter.

29. The method of claim 28, wherein the subcellular matter is mitochondria.

30. The method of claim 17, wherein the images are obtained by exposing the tissue to light having a wavelength between 700 and 900 nm.

31. The method of claim 2930, wherein the images are obtained by exposing the tissue to light having a wavelength of about 740 nm.

32. The method of claim 30, wherein the images are obtained by exposing the tissue to light having a wavelength of about 800 nm.

33. A method for analyzing tissue having at least two layers of cells comprising:
(a) obtaining a multi-photon excited fluorescence image of the tissue at each of two or more depths within the tissue;
(b) determining a power spectral density (PSD) function at each depth at a designated range of spatial frequencies, and
(c) comparing the PSD functions obtained.

34. The method of claim 33, wherein the two or more depths correspond to different cellular layers within the tissue.

35. The method of claim 33, wherein the high resolution fluorescence is multi-photon excited fluorescence.

36. The method of claim 33, wherein the high resolution fluorescence is two-photon excited fluorescence.

37. The method of claim 1, wherein the high resolution of fluorescence is 1 micron or less.

38. The method of claim 33, wherein the fluorescence is autofluorescence.

39. The method of claim 33, wherein comparing the PSD functions comprises comparing a characteristic of each PSD function.

40. The method of claim 39, wherein the characteristic of the PSD function is a power exponent α.

41. The method of claim 39, wherein the characteristic of the PSD function is variance across depth.

42. The method of claim 33, wherein the spatial frequency range corresponds to tissue matter.

43. The method of claim 33, wherein the spatial frequency range corresponds to subcellular matter.

44. The method of claim 43, wherein the subcellular matter is mitochondria.

45. The method of claim 33, wherein the images are obtained by exposing the tissue to light having a wavelength between 700 and 900 nm.

46. The method of claim 45, wherein the images are obtained by exposing the tissue to light having a wavelength of about 740 nm.

47. The method of claim 45, wherein the images are obtained by exposing the tissue to light having a wavelength of about 800 nm.

48. A method for determining whether tissue exhibits a pre-cancerous characteristic comprising:
(a) obtaining a high resolution fluorescence image of the tissue at each of two or more depths within the tissue;
(b) determining a power spectral density (PSD) function at each depth at a designated range of spatial frequency, and
(c) comparing the PSD functions obtained wherein similar PSD functions at each depth indicate whether the tissue exhibits a pre-cancerous characteristic.

49. The method of claim 48, wherein the two or more depths correspond to different cellular layers within the tissue.

50. The method of claim 49, wherein the high resolution fluorescence is multi-photon excited fluorescence.

51. The method of claim 50 wherein the high resolution fluorescence is two-photon excited fluorescence.

52. The method of claim 49, wherein the high resolution of fluorescence is 1 micron or less.

53. The method of claim 49 wherein the fluorescence is autofluorescence.

54. The method of claim 49, wherein comparing the PSD functions comprises comparing a characteristic of each PSD function.

55. The method of claim 54, wherein similar characteristics at each depth indicate whether the tissue exhibits a pre-cancerous characteristic.

56. The method of claim 54, wherein the characteristic of the PSD function is a power exponent α.

57. The method of claim 54, wherein the characteristic of the PSD function is variance across depth.

58. The method of claim 49, wherein the spatial frequency range corresponds to tissue matter.

59. The method of claim 49, wherein the spatial frequency range corresponds to subcellular matter.

60. The method of claim 59, wherein the subcellular matter is mitochondria.

61. The method of claim 49, wherein the images are obtained by exposing the tissue to light having a wavelength between 700 and 900 nm.

62. The method of claim 61, wherein the images are obtained by exposing the tissue to light having a wavelength of about 740 nm.

63. The method of claim 6261 wherein the images are obtained by exposing the tissue to light having a wavelength of about 800 nm.

64. A computer program product for analyzing a tissue having at least two layers of cells, the computer program product tangibly embodied in an information carrier and including instructions being operable to cause data processing apparatus to:
(a) determine a power spectral density (PSD) function at a range of spatial frequencies for each of two or more images of the tissue, wherein each image is obtained at a different depth within the tissue, the PSD function being determined using high resolution fluorescence images of the tissue, and
(b) compare the PSD functions obtained.

65. The computer program product of claim 64, wherein the two or more depths correspond to different cellular layers within the tissue.

66. The computer program product of claim 64, wherein the high resolution fluorescence is multi-photon excited fluorescence.

67. The computer program product of claim 64, wherein the high resolution fluorescence is two-photon excited fluorescence.

68. The computer program product of claim 64, wherein high resolution is on the order of 1 micron or less.

69. The computer program product of claim 64, wherein the fluorescence is autofluorescence.

70. The computer program product of claim 64, wherein comparing the PSD functions comprises comparing a characteristic of each PSD function.

71. The computer program product of claim 70, wherein the characteristic of the PSD function is a power exponent α.

72. The computer program product of claim 70, wherein the characteristic of the PSD function is variance across depth.

73. The computer program product of claim 64, wherein the spatial frequency range corresponds to tissue matter.

74. The computer program product of claim 64, wherein the spatial frequency range corresponds to subcellular matter.

75. The computer program product of claim 74, wherein the subcellular matter is mitochondria.

76. The computer program product of claim 64, wherein the images are obtained by exposing the tissue to light having a wavelength between 700 and 900 nm.

77. The computer program product of claim 76, wherein the images are obtained by exposing the tissue to light having a wavelength of about 740 nm.

78. The computer program product of claim 76, wherein the images are obtained by exposing the tissue to light having a wavelength of about 800 nm.

79. A computer program product for analyzing a tissue having at least two layers of cells, the computer program product tangibly embodied in an information carrier and including instructions being operable to cause data processing apparatus to:
(a) obtain at least one high resolution fluorescence image of the tissue at two or more depths within the tissue;
(b) determine a power spectral density (PSD) function at each depth at a range of spatial frequencies corresponding to subcellular matter, and
(c) compare a characteristic of each PSD function obtained at a plurality of depths.

80. The computer program product of claim 79, wherein the two or more depths correspond to different cellular layers within the tissue.

81. The computer program product of claim 79, wherein the high resolution fluorescence is multi-photon excited fluorescence.

82. The computer program product of claim 79, wherein the high resolution fluorescence is two-photon excited fluorescence.

83. The computer program product of claim 79 wherein the high resolution fluorescence is 1 micron or less.

84. The computer program product of claim 79, wherein the fluorescence is autofluorescence.

85. The computer program product of claim 79, wherein comparing the PSD functions comprises comparing a characteristic of each PSD function.

86. The computer program product of claim 85, wherein the characteristic of the PSD function is a power exponent α.

87. The computer program product of claim 85, wherein the characteristic of the PSD function is the variance across depth.

88. The computer program product of claim 79, wherein the spatial frequency range corresponds to tissue matter.

89. The computer program product of claim 79, wherein the spatial frequency range corresponds to subcellular matter.

90. The computer program product of claim 89, wherein the subcellular matter is mitochondria.

91. The computer program product of claim 79, wherein the images are obtained by exposing the tissue to light having a wavelength between 700 and 900 nm.

92. The computer program product of claim 91, wherein the images are obtained by exposing the tissue to light having a wavelength of about 740 nm.

93. The computer program product of claim 91, wherein the images are obtained by exposing the tissue to light having a wavelength of about 800 nm.
